(19) United States
(12) Patent Application Publication
(10) Pub. No.: US 2013/0344166 A1
(43) Pub. Date: Dec. 26, 2013

(54) TOPICAL MIXTURE AND METHOD FOR DERMAL APPLICATION TO REMOVE EXCESS IRON AND OTHER HEAVY METALS FROM CELLULAR TISSUE

(71) Applicant: PIERRE ERNEST LOUBET, Malibu, CA (US)

(72) Inventor: PIERRE ERNEST LOUBET, Malibu, CA (US)

(21) Appl. No.: 13/532,571

(22) Filed: Jun. 25, 2012

(51) Int. Cl. A61K 31/555; A61K 33/24; A61P 43/00; A61K 33/38; A61K 38/39

(52) U.S. Cl. 424/618; 514/188; 514/17.2; 424/646

(57) ABSTRACT

A topical mixture intended to reduce undesirable Iron levels in the cells to optimize Mitochondrial functioning. A method of reducing undesirable levels of Iron in the cells to optimize Mitochondrial functioning.

### BACKGROUND OF THE INVENTION

1. Field of the Invention

Present invention relates to a novel composition suitable for removal of excess Iron and other heavy metals from cells. The present invention also relates to a novel method for removal of undesirable levels of Iron and other heavy metals from living cells.

More specifically the present invention relates to a novel topical solution with a liquid carrier base that optimally distributes all the components of the mixture throughout the carrier base. A healthy form of trivalent Chromium; L-Histidine to facilitate production of Gastro Hydrochloric Acid; and a transdermal catalyst that facilitates delivery of the metal ridding substance through the skin into the bloodstream and eventually into the body of the cells

Even further the present invention consist of a novel method for removing excess Iron and other heavy metals from the cells of a living being by applying a topical solution externally on the skin which provides for the transdermal delivery of a desirable form of trivalent Chromium into the bloodstream and cells which removes the excess Iron, or other heavy metals, thereby facilitating healthy operation of the cells. Also included in the novel method are the steps of providing, a liquid carrier base; Histidine to control Gastro Hydrochloric Acids; a transdermal catalyst to facilitate easy delivery of the metal ridding substances through the dermis and into the bloodstream.

A further alternative for both the novel topical solution and method of the present invention is the provision of an additional ingredient intended to prevent contact dermatitis (skin rash) such as Colloidal silver, Organic Oregano oil, some form of Zinc such as Zinc Oxide, and/or Collagen.

2. Description of the Prior Art

Maintenance of a balanced level of metals in the bloodstream and in the cells in general is essential to healthy living. Of particular interest to this invention is maintenance of a healthy Iron level of the cells. Imbalance either in the form of an excess or a deficit of Iron causes severe problems. While in the theory this invention could have application to any number of living beings it is primarily directed at humans. An Iron deficit in humans causes what is known as Anemia. Until recently, less was known about problems associated with the presence of excess Iron in the blood and in other living cells

Recent learned studies have suggested that excess Iron in the cells of human beings can be associated with such maladies as Diabetes and have been linked to certain types of cancer in human patients. One such malady has been referred to as the Vikings Disease (Hemochromatosis). Current treatment for Hemochromatosis includes periodic bleeding of the patient. (NIH, Mayo Clinic) In accordance with this bleeding treatment, the patient reports to his physician's office on a periodic basis such as monthly and has a prescribed amount of blood removed from the body to achieve a healthier ratio of Iron in living tissue. This is clearly an undesirable alternative.

It is believed that the problem with excess Iron in certain individuals is related to the malfunction of the Mitochondria of the cells. For some reason certain individuals cannot process Iron in the blood, it accumulates inside the cells and it is believed that the Iron blocks or clogs the Mitochondria and accumulates in them. As we all know it is the Mitochondria that are the engines of life at the cellular level. The Mitochondria work in accordance with what is known as the Krebs cycle taking Carbohydrates and burning them, turning them into energy, Carbon dioxide, and water. When the Mitochondria do not function properly, glucose and other sugars cannot be process by the organism and this is believed to be one of the major occurrences in individuals with diabetes. Some studies (Johns Hopkins, NIH, and Mayo Clinic) have even linked excess Iron accumulation in certain cells to the incidence of cancer. Therefore, removal of the excess Iron from cells and the maintenance of healthy normal levels of Iron have long been recognized as essential to a healthy human life.

It has been recognized that substances containing trivalent Chromium have been helpful aids in facilitating proper cellular levels of Iron thereby enabling appropriate mitochondrial operation. See U.S. Pat. Nos. 4,923,855, 4,954,492 and 5,194,615. This results in the elimination of excess carbohydrates or sugars in the cells. Although the precise chemical mechanism for the operation of Chromium in this setting is not completely understood, it is known that it works. Traditionally, Chromium in the form Chromium Picolinate or Chromium Polynicotinate has been provided for ingestion with certain levels of success.

However the problem persists in many patients even when treated with tablets containing desirable trivalent Chromium. The source of this problem with the traditional mechanism for the delivery of trivalent Chromium is believed to be the undesirable interaction of gastric fluids with the Chromium making them difficult to be absorbed into the bloodstream and thus delivered to the cells, or converting them into a Chromium containing compound that cannot be utilized at the cellular level to alleviate the excess Iron. These results in a patient that is a little better but not quite better enough for healthy living.

It is understood by the inventor of this invention that the solution to the problems of the prior art is the least medically invasive form of the treatment, would be the transdermal delivery of trivalent Chromium, usually in the form of Chromium Picolinate or Chromium Polynicotinate with a solution that facilitates transdermal delivery of the substances in a fashion which minimizes interaction with gastric fluids. Accordingly, at the most basic, level the present invention supplies a liquid carrier base that optimally distributes the components of the mixture throughout, together with Chromium Picolinate or Chromium Polynicotinate. Also, the Present invention contemplates provision of a transdermal catalyst which facilitates effective delivery of the Iron ridding Chromium and/or Vanadium through the dermis in the most efficient fashion.

One of the inherent issues presented by transdermal delivery of anything is the possibility of contact dermatitis, or skin irritation. This occurs with nearly anything placed upon the skin for extended periods of time. It can be seen to occur with children through the application of even simple bandages for extended periods. Accordingly, the present invention also contemplates provision of a substance intended to minimize contact dermatitis and increase skim moisture and health. This part of the invention is addressed by adding substances such as Colloidal Silver, Organic Oregano Oil, Collagen or a Zinc compound that is healthy for the skin.

### SUMMARY OF THE INVENTION

Accordingly, an object of the present invention is to provide a novel composition for transdermal delivery into the bloodstream and ultimately the cell, to reduce undesirable levels of Iron and other metals from the cells to increase efficient and proper operation of the Cellular Mitochondria thereby eliminating undesirable levels of excess Glucose in the cells.

In accordance with the present invention, there is provided a transdermal method of delivering Trivalent Chromium into the cells of a living being to reduce excess levels of Iron and other metals to improve and optimize Mitochondrial function so as to facilitate the proper utilization of Glucose and other substances by the cells.

### DETAILED DESCRIPTION OF THE PREFERRED EMBODIMENTS

### 1. Composition

The present invention relates to a means and composition for treating excess Iron in living beings referred to as “Iron Loading”. The present invention, although suitable for a wide variety of organisms, is more particularly suited for humans. Both the method and composition of the present invention are intended for enhancing the transdermal transfer of trivalent Chromium through the skin into the bloodstream and through the cell wall of human beings to absorb excess Iron or other undesirable metals such as heavy metals and remove them from the internal structure of the cell thereby enhancing the operational characteristics of the Mitochondria. This, it is believed will improve cellular processing of various nutrients and especially sugars such as Glucose.

The present invention has more particularly, for its object, a composition that is intended as a topical mixture to be applied to the skin that will include a liquid carrier base of such a nature that the various elements of the mixture are generally uniformly distributed throughout the mixture. Complete solution is not necessary, although homogeneity is a desirable state. The test mixtures devised by the inventor included as a liquid carrier base simple body lotion that were commercially available. Although the present invention contemplates a liquid carrier base, talc could suffice as a carrier. Alternatively, a liquid or solid soap could serve as the carrier base. However, no tests have been performed using talc as the format.

Also included in the present invention is trivalent Chromium. As currently contemplated, the Chromium will be in the form of Chromium Picolinate, Chromium Polynicotinate, or Chromium Chloride. The essential element is trivalent Chromium. The choice of Chromium Picolinate, Chromium Polynicotinate, or Chromium Chloride has been made by the inventors because they are readily, commercially available in most health food stores, pharmacies, and other sources of dietary supplements. Tests have shown that trivalent Chromium when absorbed into the bloodstream and delivered to the individual cells of humans reduces the levels of excess Iron within the cells improving the functional operation of the Mitochondria. This is a desirable treatment for people suffering from Diabetes and there are currently studies, which indicate that “Iron Loading” can possibly be attached to the occurrence of cancer. A possible alternative or compliment to the Chromium of the present invention is Vanadium in the form of Vanadyl Sulfate. A combination of both Chromium and Vanadium compounds have been utilized in some tests with desirable results.

It has also been found desirable according to the present invention to provide a substance, which accelerates, facilitates, and/or controls in a time-release fashion, the transfer of the trivalent Chromium through the skin into the bloodstream and to the cells as a catalyst. Any transdermal catalyst will suffice. However, the inventors of the present invention used and considered as ideal Sodium Chloride, Caffeine, Nicotinic Acid, and lastly alcohol as the transdermal catalyst ideal to the present invention. Depending upon the concentration of the trivalent Chromium in the composition of the present invention the use of either an accelerant facilitating agent or time-release control mechanism will be chosen.

The use of the transdermal catalyst is necessary because of the nature of the skin or dermis. Generally speaking, the skin seals the body to control the outward passage of moisture and other human byproducts and at the same time prevents the inward passage of undesirable environmental elements such as bacteria, viruses, and even toxic chemicals. However, it is well known that the skin does have established absorption rates for certain types of chemicals or substances.

While consumption of various forms of trivalent chromium has been the traditional method for human intake of trivalent Chromium, there are many disadvantages to the ingestion method. The major disadvantage appears to be interaction between the Chromium with gastric juices. Gastric enzymes and stomach acids are known to react with trivalent Chromium making it more difficult to absorb and utilize at the cellular level. The obvious alternative would be to inject it but that is costly, painful, and unnecessarily medically invasive. Transdermal intake is the perfect alternative.

That is why the present invention has as one of its objects the provision of a topical solution, which permits the absorption through the skin of the trivalent Chromium necessary for healthy levels of sugars in the cells in an optimal fashion that is minimally medically invasive. It is well known that Sodium Chloride, Nicotinic Acid, Caffeine, and alcohol act as catalysts for transdermal transfers of other substances. The effect of these various catalysts on the skin is to make the membrane more permeable then it would be in their absence, to facilitate the passage of trivalent Chromium. Each has its advantages and disadvantages but such are not the subject matter of the present invention and so it will not be discussed further.

In utilization, the composition of the present invention can be applied over the entire exterior of the body. This allows for optimal absorption of the trivalent Chromium ultimately into the cells of the body. The concentration levels of the trivalent Chromium and/or Vanadium will ultimately determine the nature of the transdermal catalyst that will be utilized. With higher concentration of trivalent chromium, the catalyst, which will act in a time-release fashion, will be ideal. In that fashion there is not an excess of trivalent Chromium in the bloodstream beyond that which can be utilize efficiently by the body thereby avoiding discharge in the urine and wastage. Excess trivalent Chromium beyond that which the body can efficiently utilize will be processed and discarded in the urine. In the composition of the present invention, with lower concentrations of trivalent Chromium, a selection of Sodium Chloride or Nicotinic Acid would be more ideal because of the rate of absorption, which they permit. Alcohols although feasible as a transdermal catalyst are the least desirable in that they are lost through evaporation quickly to the ambient, and also results in dryness of the skin. Alcohols are miscible with water and readily withdraw water from the skin resulting in a dry condition, which is undesirable.

It is well known that application to the skin of nearly anything from bandages to certain lotions can cause contact dermatitis or irritation of the skin. This is a possible undesirable side effect of lotions such as the present invention. It is a known phenomenon, which occurs with, by way of example, nicotine patches utilized by smokers to aid in the quitting smoking. After extended use of the nicotine patches, many patients utilizing them develop skin irritation. For this reason it is recommended that the patients using nicotine patches place them on different locations on different days thereby allowing the skin to recover during and after treatment. However, the nicotine patches are relatively small in size and even though small, they deliver adequate medication to alleviate the desire or craving to smoke.

However, with the present invention it is contemplated that nearly the entire surface of the body will be utilized for the application of the composition of the present invention in accordance with the method of the present invention. Therefore, it has been found desirable to add an element that will prevent the contact dermatitis. The present inventors have found Colloidal Silver, Organic Oregano Oil, Collagen, various Zinc compounds and/or combinations of these enumerated elements to be desirable additions.

For reasons not exactly or completely understood, the presence of L-Histidine and amino acid in the mixture has been found to be beneficial to lowering the levels of blood sugar in patients. It is believed that the L-Histidine facilitates the production of Gastro Hydrochloric Acid in the stomach. This it is believed facilitates better digestion of food by the body it is also believed the L-Histidine acts as a catalyst to facilitate transdermal transfer. Ingested, L-Histidine is easily broken down in the stomach in to beta-alanine, which renders it relatively useless. Transdermal application avoids this issue completely. L-Histidine is readily available in most health food stores or dietary health food stores

The present invention is best practiced by application of the trivalent Chromium composition at least twice daily. Preferably the application will be made before sleep and early in the morning ideally before exercise. Although the present invention may be applied to the entire body it is recommended it be applied to the legs and groin area where easy access to a large number of blood vessels can be achieved. Additionally, the legs and groin area contained some of the most exercised muscles in the human body. An alternative carrier base can be achieved through infusion of the trivalent Chromium and other elements into a bar of soap so as to achieve even distribution over the entire body.

### 2. Formulations and Test Results

Below is set forth a typical early batch of the composition of the present invention which is utilized in accordance with the method of the present invention, to wit:

Remove 2 oz. from a 20 oz. bottle of carrier lotion . . . remain 18 parts left

| Solids | | Per dose | |
|---|---|---|---|
| 1 ts Caffeine* if needed or wanted. | 5 grms | 4 | mgrm |
| 1 ts Histidine | 5 grms | 4 | mgrm |
| 20-200 mg Chromium Picolinate tabs | 40 grms | 2 | grms |
| Nicotinic Acid | 5 grms | 4 | mgrm |
| Medial Quality Himalayan salt | 1 tbs = 4 grms | .5 | mgr |

### ***************************Liquid

Add 2 oz. colloidal Chromium Vanadium

### 1 ts Colloidal Silver for 500 ppm

Bottle contains twenty (20) one ounce dosage size . . .

### Other Ingredients

Water, Stearic Acid, Cetyl Alcohol, Glycerol Monostearate, Mineral Oil, Petrolatum, Triethanolamine, Carbomer, Tocopherl Acetate (vitamin E) Propylene Glycol, Diazolidinyl Urea, Lodopropynyl, Butylcarbamate, DMDM Hydantoin, Fragrances, Soluble Collagen.

| Active Ingredients: | Purpose |
|---|---|
| Trivalent Chromium | Normalize blood sugar, cellular energy, |
| | Reduce lactic acid production |
| Colloidal Silver | Skin health, anti-bacterial |
| Amino Acids | Supplemental Health |
| Nicotinic Acid | Dermal Facilitator |
| Sodium Chloride | Dermal Accelerant |
| Vanadium | Micronutrient |

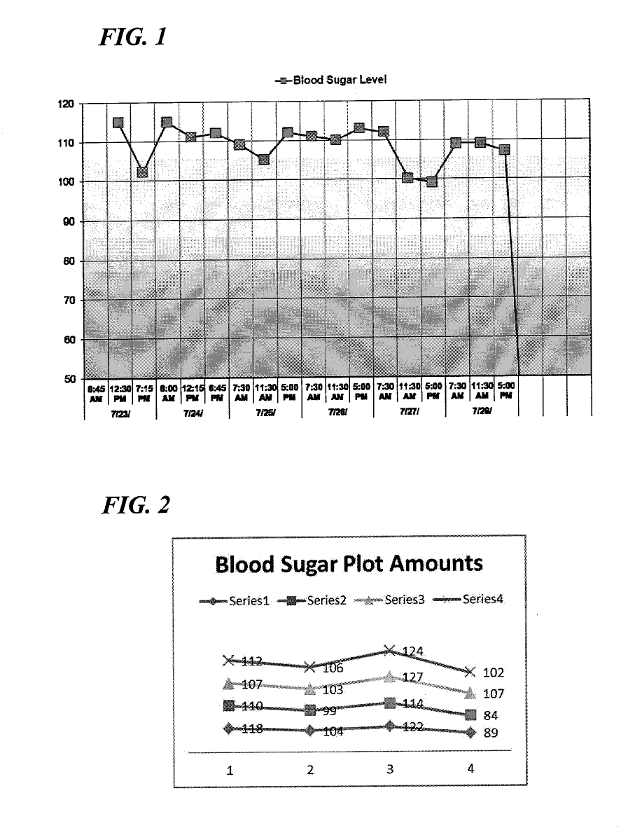

FIGS. 1 and 2 and the charts below show the blood sugar level of a patient utilizing the present invention in accordance with the formulation set above. The blood sugar level can be seen to drop dramatically after application of the present invention to the surface of the skin.

Formula 3

| | |
|---|---|
| Date | Time |

| | | | |
|---|---|---|---|
| Day 1 | 8:45 | AM | |
| | 12:30 | PM | 115 |
| | 7:15 | PM | 102 |
| Day 2 | 8:00 | AM | 115 |
| | 12:15 | PM | 111 |
| | 6:45 | PM | 112 |
| Day 3 | 7:30 | AM | 109 |
| | 11:30 | AM | 105 |
| | 5:00 | PM | 112 |
| Day 4 | 7:30 | AM | 111 |
| | 11:30 | AM | 110 |
| | 5:00 | PM | 113 |
| Day 5 | 7:30 | AM | 112 |
| | 11:30 | AM | 100 |
| | 5:00 | PM | 99 |
| Day 6 | 7:30 | AM | 109 |

Formula 7

| AM | AM after | PM | PM after | Avg. |
|---|---|---|---|---|
| 118 | 104 | 122 | 89 | 108.25 |
| 110 | 99 | 114 | 84 | 101.75 |
| 107 | 103 | 127 | 107 | 110.25 |
| 112 | 106 | 124 | 102 | 111 |

While we described in this detailed description of the preferred embodiments the best known mode of making and practicing the present invention, nothing herein should be construed to limit the invention herein except as set forth in the following claims.

Therefore, what I claim as my invention is:

1-5. (canceled)

6. A method for reducing undesirable levels of Iron or other heavy metals in cells so as to optimize Mitochondrial functioning, comprising application to the dermis of the mixture as defined in claim 1, 4, or 5.

7. Topical mixture intended for application to the skin so as to deliver substances to the bloodstream in the most efficient and effective fashion in order to optimize delivery of said substances to the interior of animal cells in order to absorb undesirable levels of iron and other heavy metals in the cells, comprising:
a. A carrier base that optimally distributes all components of the mixture throughout in a generally homogenous fashion;
b. Trivalent Chromium containing substance chosen from the group consisting of:
1. Chromium Picolinate;
2. Chromium Polynicotinate; or
3. A substance containing trivalent Chromium and Vanadium Oxide;
c. A transdermal catalyst that facilitates transfer of the trivalent Chromium through the skin and into the bloodstream so as to minimize the destructive effect of digestive enzymes upon the trivalent Chromium; and
d. A silver compound to facilitate migration of the trivalent chromium through cellular membranes to optimize effective utilization of the trivalent chromium consisting of colloidal silver; and
e. L-Histidine.

8. The topical mixture of claim 7 wherein the transdermal catalyst that facilitates transfer of the trivalent Chromium through the dermis into the bloodstream and to the interior of the cell membranes is selected from the group consisting of:
a. Ethyl Alcohol;
b. Sodium Chloride;
c. Nicotonic Acid; or
d. Caffeine.

9. The topical mixture of claim 7 including a substance intended to prevent contact dermatitis and maintain a healthy moisture level of the skin selected from the group consisting of
1. Organic Oregano Oil;
2. Collagen; or
3. a Zinc containing substance.

10. The topical mixture of claim 8 including a substance intended to prevent contact dermatitis and maintain a healthy moisture level of the skin selected from the group consisting of:
4. Organic Oregano Oil;
5. Collagen; or
6. a Zinc containing substance.
